(19) United States
(12) Patent Application Publication
(10) Pub. No.: US 2025/0246269 A1
(43) Pub. Date: Jul. 31, 2025

(54) METHOD FOR PREDICTING PROPERTIES OF ELECTROLYTES

(71) Applicant: CARNEGIE MELLON UNIVERSITY, Pittsburgh, PA (US)

(72) Inventors: Yumin ZHANG, Pittsburgh, PA (US); Imanuel BIER, Durham, NC (US); Venkatasubramanian VISWANATHAN, Pittsburgh, PA (US)

(21) Appl. No.: 18/707,434

(22) PCT Filed: Nov. 14, 2022
(86) PCT No.: PCT/US2022/049835

§ 371 Date: May 3, 2024

Related U.S. Application Data
(60) Provisional application No. 63/278,561, filed on Nov. 12, 2021.

(51) Int. Cl. G16C 20/30; G16C 10/00; G16C 60/00

(52) U.S. Cl. CPC G16C 20/30; G16C 10/00; G16C 60/00

(57) ABSTRACT

A system, method, and computer program product for predicting properties of electrolytes in a liquid electrolyte mixture independent of empirical fitting from experimentally measured properties. An example aspect is configured to: validate at least one molecular force field, prepare a liquid electrolyte mixture corresponding to the at least one molecular force field, perform molecular dynamic simulations of the electrolyte mixture, obtain simulated molecular dynamic trajectories from the molecular dynamic simulations, and perform molecular structure analysis to predict ionic conductivity, ion pairing properties, solubility properties, and/or other thermodynamic properties of the liquid electrolyte mixture.

### CROSS REFERENCE TO RELATED APPLICATIONS

This application claims priority to and the benefit of U.S. Provisional Patent Application No. 63/278,561, filed on Nov. 12, 2021, entitled METHOD FOR PREDICTING PROPERTIES OF ELECTROLYTES, which is expressly incorporated herein by reference in its entirety.

### GOVERNMENT RIGHTS

This invention was made with U.S. Government support under DE-AR0001211 awarded by the Department of Energy (DOE). The U.S. Government has certain rights in this invention.

### FIELD OF THE INVENTION

This disclosure generally relates to the field of electrolyte analysis, and more particularly, predicting properties and designing electrolytes.

### BACKGROUND

A liquid electrolyte mixture, composed of at least one solvent and one salt, is a necessary component of electrochemical devices. Liquid electrolytes have remained indispensable due to their capacity to leverage existing manufacturing lines and keep costs low. In polar solvents, regardless of aqueous or non-aqueous, electrolytes dissolve to produce oppositely charged ions as charge carriers to participate in the charging and discharging processes. In a typical rechargeable energy storage device, metal cations deposit/intercalate and dissolute/deintercalated through reactions, thereby traveling back and forth between the negative and the positive electrodes. As a result, the transport properties of charge carriers within the electrolyte mixture play an important role in determining the power performance and the charging rate of the device.

Ion pairing between the cations and the anions is a known phenomenon of liquid electrolyte mixtures and is common in all electrochemical systems. The relative populations of each type of ion pairing clusters evolve with the electrolyte concentrations, solvent properties such as dielectric constant and viscosity, and temperatures. The formation of ion pairs increases with the increasing electrolyte concentration, thereby reducing conductance. The thermodynamic properties, such as activity, activity coefficients, and equilibrium constant associated with ion pairing behavior have large impacts on an electrolyte's transport properties, as well as electrolyte property derived metal dendrite evolution.

The solubility/saturation limit is one of the basic physical properties of an electrolyte mixture. This is the electrolyte concentration limit when the ionic conductivity reaches nearly zero due to significant ion pairing. The solubility limit assists in determining the feasibility of whether a solvent molecule or its mixture can be used to accommodate high concentrated electrolyte (HCE) and localized high concentrated electrolyte (LHCE) designs. It has been shown that HCE and LHCE improve electrolyte-electrode interfacial stability, cycling performance, and electrochemical potential window for high voltage cathodes. Therefore, accurate evaluation of a solvent mixture's dissolving ability or high solubility limit before its synthesis and handling has significant merit in applications such as high concentrated ionic liquid and liquid-in-salt electrolytes for liquid metal batteries such as lithium metal batteries.

Ionic conductivity of electrolyte mixtures has previously been predicted from first principles using the Nernst-Einstein (NE) equation, or related transport property expressions. The hallmark of such equations is an expression of proportionality between the ionic conductivity of the solution and the diffusion coefficients of charge carrier species, including their concentrations. The diffusion coefficients can be obtained by experimental approaches and molecular dynamic simulations (MD). However, it is well known that the agreement between predicted ionic conductivity using such expressions and experimentally measured values is poor. This is because the NE type formalism assumes that ions and related charge carrier aggregates do not interact, which is only valid in extreme dilution, thereby incorrect for realistic energy storage applications.

Accordingly, there is a need for systems and methods to efficiently design liquid electrolyte mixtures through a reliable process that is independent of experiments for fitting purposes and applicable for high-throughput computation screening platforms to enable next-generation storage devices.

### BRIEF SUMMARY

The systems and methods of the present disclosure enable the prediction and analysis of electrolyte properties independent of empirical fitting from experimentally measured properties.

The presently disclosed systems and methods do not suffer from the limitations of the prior art by enabling direct analysis of evolving fractions of charge carriers in electrolyte mixtures, offering more accurate and more realistic predicted conductance and ionic conductivity. Thus, the systems and methods of the present disclosure may predict bulk properties that can directly inform materials screening.

The systems and methods of the present disclosure validate at least one molecular force field and prepare a liquid electrolyte mixture corresponding to the at least one molecular force field. Molecular dynamic (MD) simulations may be performed of the electrolyte mixture, wherein simulated molecular dynamic trajectories obtained from the MD simulations undergo molecular structure analysis and conductance formalism to predict ionic conductivity, ion pairing properties, solubility properties, and/or other thermodynamic properties of the liquid electrolyte mixture.

The presently disclosed systems and methods may be embodied as a system, method, or computer program product embodied in any tangible medium of expression having computer useable program code embodied in the medium.

### DESCRIPTION OF THE DRAWINGS

It is to be understood that both the foregoing summary and the following drawings and detailed description may be exemplary and may not be restrictive of the aspects of the present disclosure as claimed. Certain details may be set forth in order to provide a better understanding of various features, aspects, and advantages of the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the invention.

The present disclosure may be better understood by reference to the accompanying drawing sheets, in which:

FIG. 1 is a flow chart of a method for predicting properties of electrolytes, in accordance with certain aspects of the presently disclosed invention described herein.

FIG. 2 is a block diagram of a system for predicting properties of electrolytes, in accordance with certain aspects of the presently disclosed invention described herein.

FIG. 3 depicts typical components in a liquid electrolyte mixture for energy storage applications.

FIGS. 4A and 4B illustrate a solvation structure within a liquid electrolyte mixture in low to medium salt concentration or for cation-anion pairs that have weak to intermediate ion pairing tendency.

FIG. 4C illustrates a solvation structure within a medium to high salt concentrated liquid electrolyte blend or for cation-anion pairs that have strong ion-pairing tendency.

FIG. 5 depicts the radial distribution function as a liquid structure description for an example electrolyte mixture containing dilute to high concentrated LiPF₆ salt.

FIG. 6 depicts the radial distribution function as a liquid structure description for an example electrolyte mixture containing dilute to high concentrated LiFSI salt.

FIGS. 7A and 7B show the ionic conductivity calculated using the Nernst-Einstein equation and experimental conductivity measurements for example liquid electrolyte mixtures containing LiPF₆ as the salt and LiFSI as the salt, respectively.

FIG. 8A shows the agreement between the predicted and experimental ionic conductivity for an example liquid electrolyte mixture containing LiPF₆ as the salt using the systems and methods of the present disclosure.

FIG. 8B shows the agreement between the predicted and experimental conductance for an example liquid electrolyte mixture containing LiPF₆ as the salt using the systems and methods of the present disclosure.

FIG. 8C shows the associated fraction of charge carriers obtained from analyzing the molecular dynamic simulations results.

FIG. 8D shows the derived activity coefficients for the LiPF₆ system.

FIG. 9A shows the agreement between the predicted and experimental ionic conductivity for an example liquid electrolyte mixture containing LiFSI as the salt using the systems and methods of the present disclosure.

FIG. 9B shows the agreement between the predicted and experimental conductance for an example electrolyte mixture containing LiFSI as the salt using the systems and methods of the present disclosure.

FIG. 9C shows the associated fraction of charge carriers obtained from analyzing the molecular dynamic simulations results.

FIG. 9D shows the derived activity coefficients for the LiFSI system.

### DETAILED DESCRIPTION

This disclosure generally describes systems and methods of predicting and analyzing electrolyte properties independent of empirical fitting from experimentally measured properties.

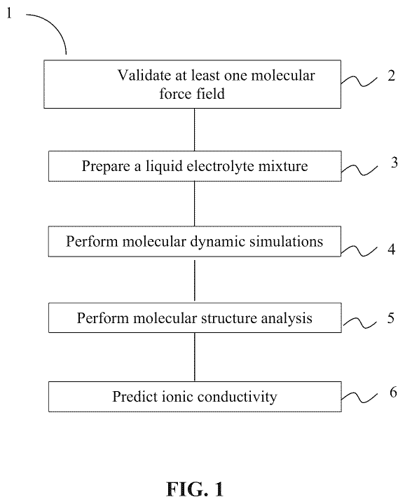

The present disclosure provides a method 1 (FIG. 1) for predicting and analyzing properties of electrolytes. The method 1 may validate at least one molecular force field 2 and prepare a liquid electrotype mixture 3 corresponding to the at least one molecular force field 2. The method 1 may validate at least one molecular force field by fitting the parameters using methods known in the art or by using any machine learned inter-atomic potential. In some aspects, the force fields may include nonpolarizable force fields and/or polarizable force fields. The method 1 may select an appropriate force field using the atomic and bonding properties within a molecule and its long-range and short-range interactions with other molecules in an electrolyte mixture. An accurate molecular force field may increase the reliability of the predicted properties. The systems and methods of the present disclosure may validate the at least one molecular force field by performing a benchmark of the at least one molecular force field against experimental or accurate electronic structure theory (including but not limited to, Density Functional Theory, Hartree-Fock Theories, etc.) dipole moments, and dielectric constants and densities of pure solvents and electrolyte mixture to compare with simulated properties to determine whether there is agreement within a reasonable discrepancy. A molecular force field of the presently disclosed systems and methods may include, but is not limited to OPLS and its variants (OPLS 3, OPLS 3e, OPLS4, etc.), AMBER, CHARMM, OpenFF, and APPLE&P.

After validating at least one molecular force field 2, the method 1 may then prepare a liquid electrolyte mixture 3 corresponding to the at least one validated molecular force field 2. According to certain aspects, the liquid electrolyte mixture 3 may cover a range of relatively dilute salt concentrations to relatively concentrated salt concentrations. In one aspect, the liquid electrolyte mixture 3 may be prepared by first mixing the desired solvents followed by an MD equilibration process. The metal cations and anions may then be added to the MD equilibrated solvents. This may permit quicker equilibration than adding the solvents, cations, and anions all at once. In other aspects, further equilibration of the mixture containing the solvents and salts may be performed until a desired equilibrium is reached.

The method 1 may then simulate the liquid electrolyte mixture by Molecular Dynamics 4 to generate dynamical data. Dynamical data includes, but is not limited to, molecular dynamic trajectories. Simulations may be performed for a sufficiently long time to produce simulated molecular movement trajectories, which may be further analyzed using the systems and methods of the present disclosure.

More specifically, a liquid electrolyte mixture simulated by MD will contain multiple types of solvation structures, wherein each type of solvation structure is composed of solvent and ion components. With increasing salt concentration, the probability of observing different cation-anion pairing phenomena is higher, and the probability of separated ion pairs is lower.

The method 1 may perform molecular structure analysis of the simulated molecular dynamic trajectories obtained from the MD simulations 5. Performing molecular structure analysis 5 may include determining the types of molecules in the system, wherein the types of molecules are determined automatically or using a topology file. According to some aspects, the topology file defines which atoms are connected to one another through chemical bonds. Performing molecular structure analysis 5 then may determine which atoms are bonded as well as which atoms make up each molecular unit in the simulation. In some aspects, there may be tens of thousands of atoms and thousands of molecular units comprised of those atoms.

The systems and methods of the present disclosure may be used in liquid electrolyte applications for metal batteries, including but not limited to, high concentrated ionic liquid and liquid-in-salt electrolyte mixtures. For each metal atom in the simulation, the solvation structure may be determined by finding all atoms within a radius of the metal atom with respect to the periodic unit cell of the calculation. In some aspects, the radius may be the van der Waals radii of the metal atom (cation/anion) multiplied by a factor of 0.9 to 1.2, such as 1.1. For each atom within the radius of the metal atom, the entire molecule containing each atom may be considered to be in the solvation structure. Aggregated structures may be further defined and resolved by combining solvation structures if a molecule is shared by the solvation structure of two or more metal atoms. The number of stoichiometric solvation structures may be tabulated and summarized as averages from the simulated molecular dynamic trajectories.

The molecular trajectories may then be analyzed to determine solvation structures. The molecular trajectories may be analyzed for any relevant time period, such as for at least 5 nanoseconds, 10 nanoseconds, 15 nanoseconds, 20 nanoseconds, or more. The molecular trajectories may be analyzed to equilibrium of the solvation structure, such as up to 50 nanoseconds or more. For example, the trajectories may be analyzed for at least 10 nanoseconds for strong electrolytes. Strong electrolytes include, but are not limited to, strong acids such as HCl, HBr, HI, HNO₃, HClO₃, HClO₄, and H₂SO₄, strong bases such as NaOH, KOH, LiOH, Ba(OH)₂, and Ca(OH)₂, and salts such as NaCl, KBr, and MgCl₂. The trajectories may be analyzed for at least 20 nanoseconds for weak electrolytes. Weak electrolytes include, but are not limited to, weak acids such as HF, HC₂H₃O₂, H₂CO₃, and H₃PO₄ and weak bases such as NH₃ and C₅H₅N. The trajectories may be analyzed for 20 to 50 nanoseconds to reduce uncertainty in the resulting solvation structures.

The metal atom may comprise any metal or metal alloy having a low melting temperature. Exemplary metals include lithium, gallium, cesium, rubidium, francium, and alloys thereof. The metal alloy may comprise a sodium-potassium alloy. A preferred metal is lithium.

The methods and systems of the present disclosure may conduct solvation cluster analysis and provide the probability densities of each cluster species. Thus, the systems and methods of the present disclosure may determine the probability densities of the solvation cluster composition and may select the best electrolyte concentrations based on the experimental molarity of the highest ionic conductivity observed.

Solvation structures include, but are not limited to, solvent-separated ion pairs (SSIP), contact-ion pairs (CIP), aggregates (AGG). Solvation structures as used herein include, but are not limited to, labile solvation structures wherein the solvation structures may form, dissipate, and evolve throughout ranges of salt concentration. Therefore, the methods and systems of the present disclosure may analyze the different types of solvation structures as well as the composition of the solvent to determine optimal electrolyte concentration ranges in a liquid electrolyte mixture.

The simulated molecular dynamic trajectories obtained from the molecular dynamic simulations 4 may be analyzed through molecular structure analysis 5 and efficient statistical sampling to obtain the time-averaged fractions of charge carries (“a”) in solution. Charge carriers according to aspects of the present disclosure may be ion pairing clusters that have a net charge. Analysis on the molecular clustering lifetime may be conducted to ensure that sufficient time is included in the molecular trajectory data.

According to certain aspects, liquid structure analysis utilizing radial distribution function (RDF) analysis may obtain a quantity “a”, wherein “a” denotes the size of the ionic atmosphere. “a” is a physics-based quantity that is unique to any specific set of metal cation-anion pairs and is dependent upon the composition of the solvent mixture.

After performing molecular structure analysis 5 of the simulated molecular dynamic trajectories, the method 1 may predict the ionic conductivity 6 of the liquid electrolyte mixture 3. The methods and systems of the present disclosure may apply the Nernst Equation (NE), when the theory is valid, to extrapolate the conductance at infinite dilute (Λ°) from the calculated ionic conductivity at extreme dilute concentrations. Therefore, the NE equation may be suitable for obtaining conductance and ionic conductivity at extreme dilute situations where ion-ion interactions are negligible, wherein the systems and methods of the present disclosure are suitable for obtaining conductance and ionic conductivity at any reasonable concentration whether it is dilute or saturated.

The systems and methods of the present disclosure may further perform conductance formalism of data obtained from the molecular structure analysis and predict the conductance of the liquid electrolyte mixture. Quantities a, Λ°, and the fractions a of charge carriers for liquid electrolyte mixtures at a wide range of concentrations may be used to compute the conductance of the liquid electrolyte mixture using a formalism previously developed in the art by the equation:

$$Λ = Λ° - S √ α c + E \times α c \times log ( α c ) + J ( α c ) - {K}_{A} {Λγ}^{2} \times α c \qquad EQ . 1$$

wherein Λ is conductance, Λ° is the conductance at infinite dilution, and S is a function of the solvent's dielectric coefficient ϵᵣ, the viscosity η, the charge number of the ion, and the temperature. E originates in the expansions of exponential polynomials containing the description on ionic atmospheres in a specific solvent mixture. J is a function of ac and depends on the theoretical treatments employed on the conductance equation and is responsible for the long-range interaction. K_{A} is the ion pairing equilibrium constant. γ is the activity coefficient of ion pairing thermodynamics. ac is the ionic strength, which is the product of the fraction of charge carriers or the salt molarity dependent fraction of free ions (a) and the ion concentration (c). In the prior art, Λ°, a, and a are unknowns to be resolved but are experimentally inaccessible. Therefore, the prior art must apply empirical fitting.

However, Λ°, a, and a may be obtained from the systems and methods of the present disclosure. The conductance and ionic conductivity may be obtained using EQ. 1, and the systems and methods of the present disclosure. The ion pairing thermodynamic quotient and the associated activity coefficients are calculated from K_{A} and the Debye-Hückel equation. The effective solubility limit may be determined by the concentration in which the ionic conductivity reaches zero.

In the prior art, the knowledge of K_{A} is essential for the derivation of a and a, which are required inputs in EQ. 1. The limitation of any other prior art using EQ. 1 and related equations comes from the assumption of a single K_{A} across the entire concentration range. The prior art has discussed that the fraction of charge carriers, rising from ion pairing phenomena, is non-linear and likely to contain multi-stage reactions. Therefore, the consideration of a single K_{A} is incorrect and leads to inaccurate predictions. The use of EQ. 1 type formalism is not recommended in the prior art due to the experimental difficulties in measuring K_{A} and the inherent inaccuracy in deriving a and a from such measurements.

The prior art has relied on experimental measurements to fit empirical parameters or the use of calculated standard reference states from which further extrapolation is employed using chemical-physics and thermodynamic formalisms. Furthermore, such previous methods have not been proven to be capable of being applied to electrolyte mixtures that have not yet been invented or synthesized. The prior art is also not capable of processing dynamic molecular interactions on a minimum timescale of at least tens of nanoseconds, which is the timeframe for known molecular processes of chemical and physical significance for ion transport. Therefore, the dependence on previous experiments currently limits the application of previous chemical-physics models to only tens of electrolytes and salts. The presently disclosed systems and methods do not depend on any extrapolation as inputs for the chemical-physics model, and inputs are directly calculated using the systems and methods of the present disclosure.

Compared to the prior art, the systems and methods of the present disclosure do not suffer from the limitation of defining the concentration ranges in each ion pairing reaction steps and do not require the experimental measurements of the ion pairing equilibrium quotient (K_{A}) or any other property. The systems and methods of the present disclosure enable direct analysis of the evolving fractions of charge carriers in electrolyte mixtures, offering more accurate and more realistic predicted conductance and ionic conductivity.

Currently, the prior art only draws qualitative or already well-known conclusions about structure-based design of liquid electrolytes. Therefore, greater success may be achieved using the systems and methods of the present disclosure, which may predict bulk properties that can directly inform materials screening.

The systems and methods of the present disclosure may also analyze the liquid structure and ion pairing properties of the molecular dynamic simulations and predict the ion pairing thermodynamic properties of the liquid electrolyte mixture. Macroscopic transport properties, such as ionic conductivity, depend on the intermolecular interactions of molecular species in the liquid electrolyte mixture. The methods and systems of the present disclosure may examine liquid electrolyte structures over a wide range of salt concentrations, wherein quantitative descriptions connect the molecular properties with the ionic conductivity.

The ionic conductivity (specific conductance), conductance (molar conductivity), and the charge carriers' diffusion coefficients are the general quantities used to describe the transport properties. Ionic conductivity is expressed as the product of the molarity and the conductance.

Thus, the systems and methods of the present disclosure may predict ionic conductivity or conductance with experimental accuracy using parameters derived from molecular dynamic simulations. In certain aspects, the method 1 performs molecular dynamic simulations 4, performs molecular structure analysis 5 by analysis of the liquid structure and ion pairing properties, and predicts ionic conductivity 6, conductance, ion pairing thermodynamic quantities, and solubility properties.

The systems and methods of the present disclosure do not suffer from the limitations associated with the constrained validity at extreme dilutions, or the unproven reliability in tracing ion clusters' diffusivities in Nernst-Einstein and related approaches. The systems and methods of the present disclosure do not require, but may include if desired, experimental measurements or extrapolation of ion pairing equilibrium quotients. The systems and methods of the present disclosure do not require fitting empirical parameters using experimentally measured ionic conductivity or any related experimental data. Therefore, the systems and methods of the present disclosure may enable the development of novel liquid electrolyte mixtures even before their synthesis. Furthermore, the systems and methods of the present disclosure may be used to accommodate high concentrated electrolyte and localized high concentrated electrolyte designs with improved stability, performance, and electrochemical potential. Thus, successful implementation of the systems and methods of the present disclosure may accelerate the development cycle for energy storage applications.

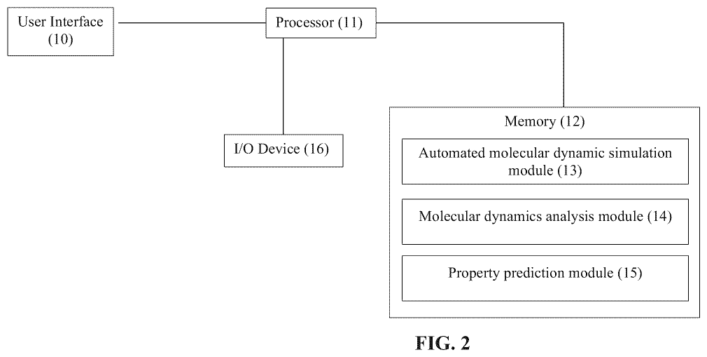

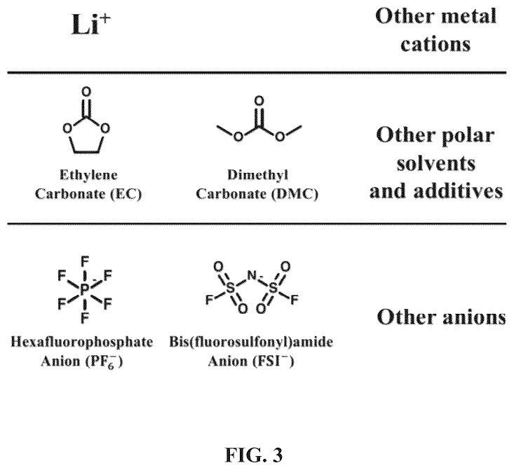

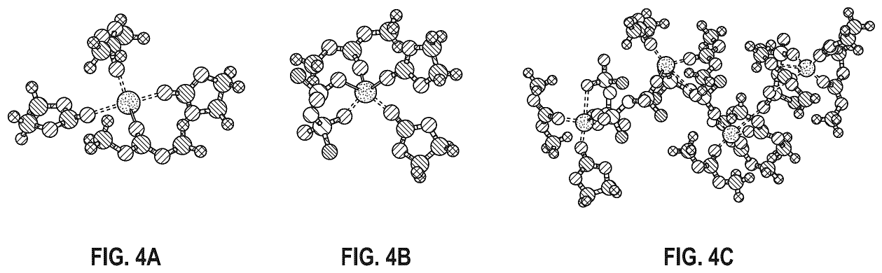

FIG. 2 is a block diagram that schematically illustrates a system according to one aspect of the present disclosure. The system may comprise a processor 11 that interfaces with memory 12 (which may be separate from or included as part of processor 11). The memory 12 may also employ cloud-based memory. In one aspect, the system may connect to a base station that includes memory and processing capabilities. The system may further comprise an I/O device 16.

The present disclosure provides systems for predicting properties of electrolytes in a liquid electrolyte mixture. Memory 12 has stored therein a number of routines that are executable by processor 11. The processor 11, in communication with the memory 12, may be configured to execute an electrolyte prediction assistant.

The electrolyte prediction assistant may comprise of an automated molecular dynamic simulation module 13, which comprises program instructions executable by processor 11 to perform molecular dynamic simulations of a liquid electrolyte mixture according to the methods of the present disclosure.

In one aspect, the automated molecular dynamic simulation module 13 may comprise program instructions executable by processor 11 to access a database comprising individual molecular force fields. The automated molecular dynamic simulation module 13 may further comprise program instructions executable by processor 11 to validate at least one individual molecular force field. According to certain aspects, the automated molecular dynamic simulation module 13 may comprise program instructions executable by processor 11 to instruct a user to prepare a liquid electrolyte mixture corresponding to at least one validated molecular force field according to the systems and methods of the present disclosure.

The electrolyte prediction assistant may comprise a molecular dynamics analysis module 14, which comprises program instructions executable by processor 11 to receive molecular dynamic simulations data and analyze ion pairings and associated properties which include, but are not limited to, conductance, ionic conductivity, and solvation structure using the methods of the present disclosure. In one aspect, the molecular dynamics analysis module 14 may access the molecular dynamic trajectories obtained from the automated molecular dynamic simulation module 13. The molecular dynamics analysis module 14 may determine the types of molecules in the liquid electrolyte mixture automatically. In one aspect, the molecular dynamics simulation module may determine the types of molecules by accessing a topology file, wherein the topology file may define atoms and their connectivity.

The electrolyte prediction assistant may comprise a property prediction module 15, which comprises program instructions executable by processor 11 to perform conductance formalisms of the ion pairings and predict transport, thermodynamic, and solubility properties of the liquid electrolyte mixture according to the methods of the present disclosure.

Processor 11 may be one or more microprocessors, microcontroller, an application specific integrated circuit (ASIC), a circuit containing one or more processing components, a group of distributed processing components, circuitry for supporting a microprocessor, or other suitable processing device that interfaces with memory 12. Processor 11 is also configured to execute computer code stored in memory 12 to complete and facilitate the activities described herein.

I/O device 16 (including, but not limited to, keyboards, displays, pointing devices, DASD, tape, CDs, DVDs, thumb drives and other memory media, etc.) may be coupled to the system either directly or through intervening I/O controllers. Network adapters may also be coupled to the system to enable the data processing system to become coupled to other data processing systems or remote printers or storage devices through intervening private or public networks. Modems, cable modems, and Ethernet cards may be just a few of the available types of network adapters.

As will be appreciated by one skilled in the art, the present invention may be embodied as a system, method, or computer program product. Accordingly, the present invention may take the form of an entirely hardware embodiment, an entirely software embodiment (including firmware, resident software, micro-code, etc.), or an embodiment combining software and hardware aspects that may all generally be referred to herein as a “system.” Furthermore, the presently disclosed invention may take the form of a computer program product embodied in any tangible medium of expression having computer useable program code embodied in the medium.

Any combination of one or more computer useable or computer readable medium(s) may be utilized. The computer-useable or computer-readable medium may be, for example but not limited to, an electronic, magnetic, optical, electromagnetic, infrared, or semiconductor system, apparatus, device, or propagation medium. Computer-readable medium may also be an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CDROM), an optical storage device, a transmission media such as those supporting the Internet or an intranet, a magnetic storage device, a digital versatile disk (DVD), a memory stick, a floppy disk, a mechanically encoded device such as punch-cards or raised structures in a groove having instructions recorded thereon, and any suitable combination of the foregoing. Note that the computer-useable or computer-readable medium may be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory. In the context of this document, a computer-useable or computer-readable medium may be any medium that can contain, store, communicate, propagate, or transport the program for use by or in connection with the instruction execution system, apparatus, or device. The computer-useable program code may be transmitted using any appropriate medium, including but not limited to wireless, wireline, optical fiber cable, RF, etc.

Computer program code for carrying out operations of the presently disclosed invention may be written in any combination of one or more programming languages. The programming language may be, but is not limited to, object-oriented programming languages (Java, Smalltalk, C++, etc.) or conventional procedural programming languages (“C” programming language, etc.). The program code may execute entirely on a user's computer, partly on the user's computer, as a stand-alone software package, partly on a user's computer and partly on a remote computer, or entirely on the remote computer or server. In the latter scenario, the remote computer may be connected to the user's computer through any type of network, including a local area network (LAN) or a wide area network (WAN), or the connection may be made to an external computer, which may include through the Internet using an Internet Services Provider. In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

The systems and methods of the present disclosure may process data on any commercially available computer. In other aspects, a computer operating system may include, but is not limited to, Linux, Windows, UNIX, Android, or MAC OS. In one aspect of the present disclosure, the forgoing processing devices or any other electronic, computation platform of a type designed for electronic processing of digital data as herein disclosed may be used.

Aspects of the present disclosure are described with reference to flowchart illustrations and/or block diagrams of methods, systems, and computer program products according to aspects of the present disclosure. It will be understood that each block of the flowchart illustrations and/or block diagrams, and combination of blocks in the flowchart illustrations and/or block diagrams, may be implemented by computer program instructions. These computer program instructions may be provided to a processor of a general purpose computer, special purpose computer, or other programmable data processing apparatus to produce a machine, which the instructions execute via the processor of the computer or other programmable data processing apparatus allowing for the implementation of the steps specified in the flowchart and/or block diagram blocks or blocks.

Various embodiments of the present disclosure may be implemented in a data processing system suitable for storing and/or executing program code that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Computer readable program instructions described herein may be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, among others.

### Definitions

Unless otherwise defined, all terms (including technical and scientific terms) used herein have the same meaning as commonly understood by one of ordinary skill in the art to which this disclosure belongs. As such, terms, such as those defined by commonly used dictionaries, should be interpreted as having a meaning that is consistent with their meaning in a context of a relevant art and should not be interpreted in an idealized or overly formal sense unless expressly so defined herein.

As used herein, the term “force field” refers to the combination of a mathematical formula and associated parameters that are used to describe the forces between atoms in a molecule and atoms/molecules in a solution and calculate the potential energy of a atoms/molecules and systems comprising the same.

As used herein, the term “and/or” includes any and all combinations of one or more of the associated listed items. Likewise, as used in the following detailed description, the term “or” is intended to mean an inclusive “or” rather than an exclusive “or.” That is, unless specified otherwise, or clear from context, “X employs A or B” is intended to mean nay of the natural inclusive permutations. Thus, if X employs A; X employs B; or X employs both A and B, then “X employs A or B” is satisfied under any of the foregoing instances.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” machine part may comprise one or more parts, and the like.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

Aspects of the present disclosure may be described herein with reference to flowchart illustrations and/or block diagrams of methods, apparatus (systems), and computer program products according to aspects of the disclosure. It will be understood that each block of the flowchart illustrations and/or block diagrams, and combinations of blocks in the flowchart illustrations and/or block diagrams, may be implemented by computer readable program instructions. The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The flowchart and block diagrams in the Figures illustrate the architecture, functionality, and operation of possible implementations of systems, methods, and computer program products according to various aspects of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, may be implemented by special purpose hardware-based systems that perform the specified functions or acts or carry out combinations of special purpose hardware and computer instructions.

Words such as “then,” “next,” etc. are not intended to limit the order of the steps; these words may be simply used to guide the reader through the description of the methods. Although process flow diagrams may describe the operations as a sequential process, many of the operations may be performed in parallel or concurrently. In addition, the order of the operations may be re-arranged. A process may correspond to a method, a function, a procedure, a subroutine, a subprogram, etc. When a process corresponds to a function, its termination may correspond to a return of the function to the calling function or the main function.

In the description, certain details are set forth in order to provide a better understanding of various embodiments of the systems and methods disclosed herein. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

Furthermore, when this disclosure states that something is “based on” something else, then such statement refers to a basis which may be based on one or more other things as well. In other words, unless expressly indicated otherwise, as used herein “based on” inclusively means “based at least in part on” or “based at least partially on.”

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

All documents cited herein may be incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that may be within the scope of this application.

### EXAMPLES

EXAMPLE 1: Liquid electrolyte mixtures LiPF₆ and LiFSI were prepared using the systems and methods of the present disclosure, with lithium as the metal cation and ethylene carbonate (EC) and dimethyl carbonate (DMC) as the solvents (1:1 by mol) as shown by the structures in FIG. 3.

Prior to molecular dynamic simulations, a benchmark of the molecular forcefields was performed against experimental dipole moments, dielectric constants, densities of pure solvents, and electrolyte mixture densities. It was found that the simulated properties agreed with the experimental measurements with reasonable discrepancy. The simulated electrolyte structures were further benchmarked by comparing the Li—O radial distribution functions and coordination numbers with results obtained using polarizable force fields. There were agreements, further demonstrating that the limitation of nonpolarizable to polarizable force fields was transferability but not reliability.

The liquid electrolyte structure evolution was investigated in a wide range of salt concentrations and found sharp and gradual changes in LiPF₆ and LiFSI-containing systems, respectively. Solvation cluster analysis was performed to determine the probability densities of each cluster species.

The parameter a is defined as the distance between Li ions obtained from the analysis of the Li⁺—Li⁺ radial distribution function (RDF) at high concentrations where large ion paired aggregates formed. Thus, the approach includes the effects of the cation and anion's geometric features. The degree of dissociation a at each concentration was obtained by analyzing the fraction of SSIP. This approach was based on the general assumption that solvation clusters involving paired ions (Li⁺-anion⁻) do not contribute as significantly to conductivity in normal experimental conditions due to their charge neutrality.

Conductance is a function of salt concentration and appears linear as a function of molarity at high dilution. This behavior is described in Ostwald dilution law (ODL), given by

$$\frac{1}{Λ} = \frac{1}{Λ°} + \frac{Λ}{{( Λ° )}^{2}} \frac{c}{{K}_{A}} \qquad EQ . 2$$

wherein the intercept may be converted to Λ° through linear regression of conductance data.

Λ° is calculated from the Nernst-Einstein (NE) conductance

$${Λ}_{NE} = \frac{{σ}_{NE}}{c} ,$$

where σ_{NE} is given by

$${σ}_{NE} = \frac{{e}^{2}}{{Vk}_{B} T} ( {N}_{+} {z}_{+}^{2} {\overline{D}}_{+} + {N}_{-} {z}_{-}^{2} {\overline{D}}_{-} ) \qquad EQ . 3$$

Terms e and k_{B} are the elementary charge and the Boltzmann constant, respectively. V and T represent the system's volume and temperature. N_{±} and z_{±} are anion and Li⁺ particle number and charge number respectively. D_{±} is the self-diffusion coefficient of ions, which were obtained by mean-square-displacements (MSD) from MD simulation at dilute concentrations. The solvent's dielectric constant ϵᵣ and viscosity n that are employed in the FOHFJ formalism were obtained from MD simulations. For dielectric constant and viscosity values of the EC:DMC 1:1 by mol mixtures, the experimentally measured data at 298K and 1 bar (ϵᵣ=31.41 and η=0.0121 poise) were used.

Molecular dynamic simulations were performed using the GROMACS 2018 program and OPLS-AA force fields. Multiple replica simulations were performed for each Li salt concentration to ensure statistical accuracy. The force field parameters for solvent molecules and ions were obtained from the LigParGen Server and literature. The atomic partial charges for EC and DMC were scaled 85% and 90% respectively to prioritize density agreements such that the simulated results for pure solvents and electrolyte mixtures were within 0.01 g/cm³ difference to experimental measurements, followed by being as close as possible for the solvent's dielectric constants and dipole moments. The fraction of SSIP was determined using tools available in the molecular crystal simulation environment software (mcse).

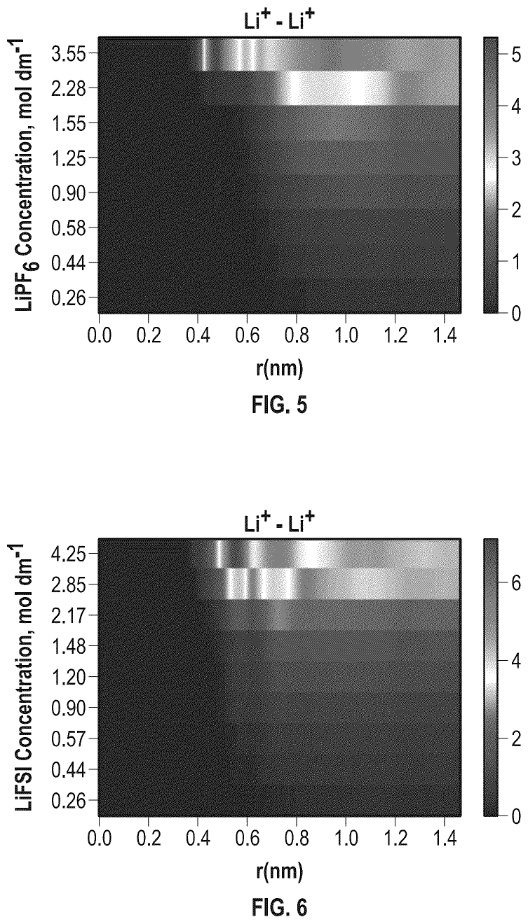

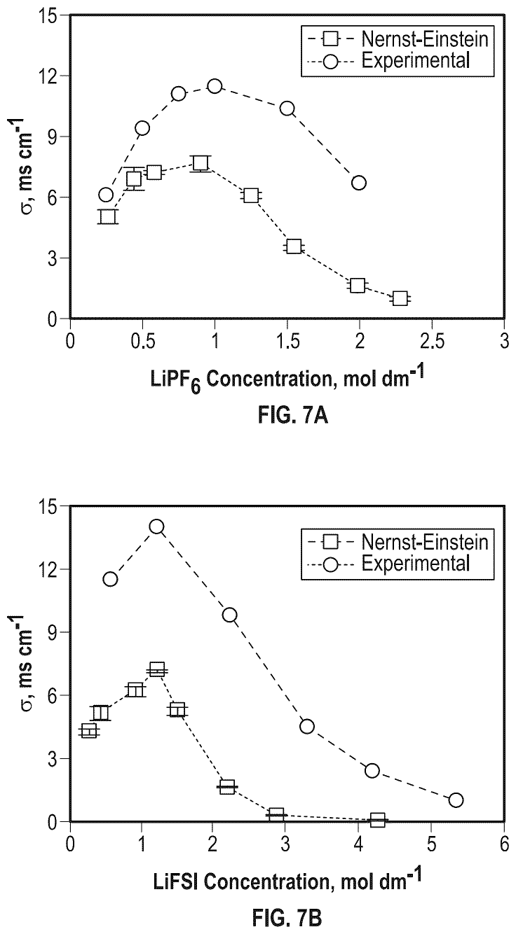

FIG. 5 demonstrates the radial distribution function as a liquid structure description obtained from the classical molecular dynamic simulations for 0.26-3.55 mol dm⁻¹ LiPF₆ in EC/DMC (1:1 by mol) solvents. FIG. 6 demonstrates the radial distribution function as a liquid structure description obtained from the classical molecular dynamic simulations for 0.26-4.25 mol dm⁻¹ LiFSI in EC/DMC (1:1 by mol) solvents. Repeating patterns were indicative of ion pairing aggregates.

To investigate the solvation structures, 50 ns NVT production simulations were performed from which simulated properties were extracted at each concentration. On the basis of the RDFs at high concentrations, an approximated closest distance that encloses an anion and a cation as the a parameter was determined. FIGS. 5 and 6 show results for LiPF₆ and LiFSI electrolyte blends from 0.26 mol dm⁻¹ to 3.55 mol dm⁻¹ and 0.26 mol dm⁻¹ to 4.25 mol dm⁻¹ (equivalent to molarity “M”), respectively. The higher Li salt concentration bound was determined when the closest Li+-Li+ peak distance ceased decreasing but plateaued. Concentrations below 0.26 M were not included since their RDF changes negligible.

The RDFs for LiPF₆ were normalized. Similar liquid structures were observed from 0.44 M to 2.28 M, and the closest Li⁺—Li⁺ distance shifted from 1.15 nm to approximately 0.9 nm. This reduction on Li⁺—Li⁺ distance may be a reflection of the increasing salt molarity. Starting from 3.55 M, drastic liquid structural changes with distinct repetitive patterns were observed. The RDF result in the range 4.73-9.78 M suggested the closest Li⁺—Li⁺ distance locates around 0.43 nm, which was applied as the a parameter. The second closest distance was approximately 0.61 nm, which attributed to an alternative local binding geometry in the primary Li⁺—Li⁺ shell. Finally, the secondary shell started around 0.95 nm, and the third was outside the r range of interest.

The RDFs for LiFSI electrolyte blends were normalized. The liquid structure evolution was more gradual compared to the LiPF₆ systems. The closest Li⁺—Li⁺ distance started around 1.15 nm at 0.44 M and shifted towards a lower distance as concentration increased. In addition, two peaks with Li⁺—Li⁺ distances ˜0.6 nm and ˜0.7 nm emerged starting from 0.9 M, and became obvious at 2.17 M. Like LiPF₆, this separation of less than 0.2 nm was attributed to different binding geometries (e.g., monodentate and bidentate) within the primary solvation shell. The second layer of ion-pairing started to show from 3.25 M with the corresponding Li⁺—Li⁺ distance at approximately 1 nm. At the highest concentration (7.56 M) sampled, the short-range ordering containing approximately two layers of paired ions became obvious. The closest Li⁺—Li⁺ distance was determined to be 0.56 nm and was later used as the a parameter for LiFSI systems. The formation of repeating peaks and valleys in the RDF at high salt concentrations was an indication of long-range aggregation, which may be considered precursors for precipitation.

Analysis of RDF gave information of the averaged liquid structure. However, it did not offer detailed information of the statistical composition of the primary solvation shell as it evolves across salt concentrations. The primary solvation cluster types and their corresponding probability densities at each concentration were analyzed according to the systems and methods of the present disclosure. The fraction of SSIPs, associated with the degree of dissociation, was obtained by summing the probability densities of all solvation clusters with 0 anions.

Though the highest ionic conductivities were similar for LiPF₆ and LiFSI, the solvation environments were different due to the corresponding electrolyte strength. “#S #A” represents the cluster composition, where “#S” and “#A” are the number of solvents and anions respectively. The most probable cluster 4SOA had a probability density of 0.68 out of 1. The clusters 5SOA and 3SIA were equally probable and had probability densities of 0.13 out of 1. Five different SSIP compositions were summed up to 0.82 as total SSIP fraction of all solvation clusters identified. CIPs and AGGs were the minority clustering species in 1.05 M LiPF₆ electrolyte blends.

Compared to LiPF₆, LiFSI favored the formation of CIPs and AGGs, making SSIPs a minority. This behavior may be rationalized by PF₆⁻ being a weaker electron donor than FSI. According to Gutmann's concept of “donor-acceptor”, PF₆⁻ was expected to show a lower tendency to interact with an acceptor, in this example, a Li⁺ ion, leading to fewer CIPs and AGGs. The total fraction of FSI containing CIPs, consisting of 2SIA, 3SIA, 4SIA, and 5SIA, occupied more than half of the identified solvation clusters. The diversity of AGGs, ranging from solvation clusters that involve two to four anions, increased more than three times compared to LiPF₆. The summation of all SSIPs gave 0.26, which represents the degree of dissociation for LiFSI at 1.2M, was used to calculate the conductance.

Further, the solvents' SSIP compositions were resolved to probe into the participation of EC and DMC in the primary solvation shells. Comparing the types of SSIPs, the distribution of the solvation clusters was similar for both LiPF₆ and LiFSI systems at their experimental highest conductivities. The most common SSIP coordination number was consistently four for both Li salt systems.

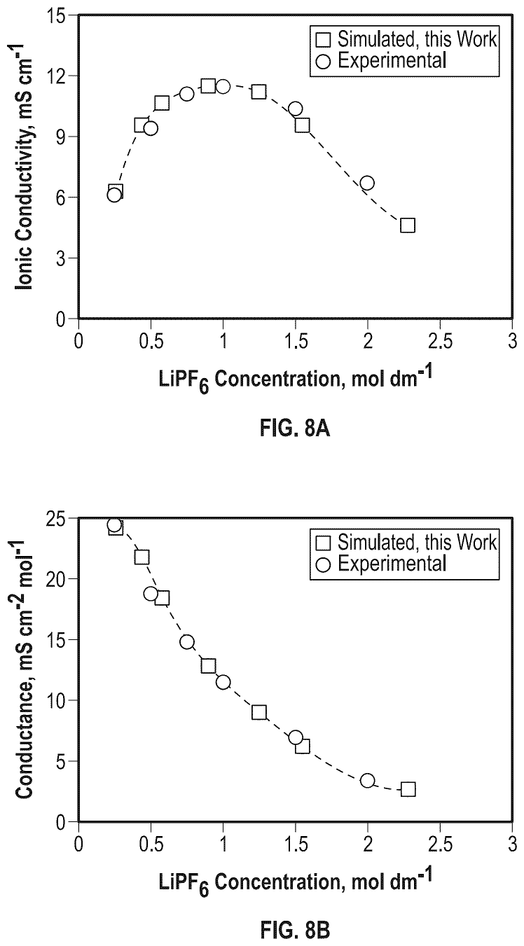

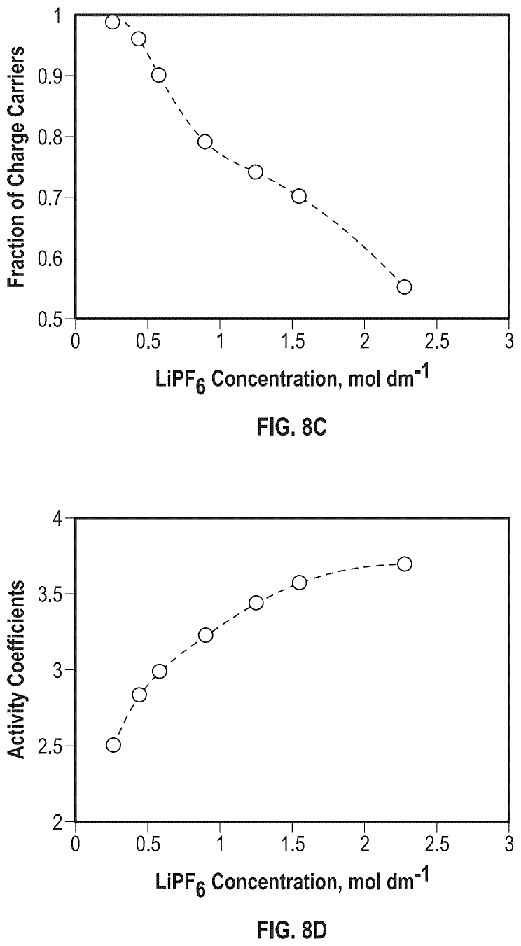

The fractions of SSIPs for the LiPF₆ electrolyte blends in the range of 0.14 M to 2.88 M and for the LiFSI blends ranging from 0.13 M to 5.79 M are shown in FIG. 8C & 9C, respectively. These concentration ranges were chosen based on the availability of the experimental data for comparison purposes. The largest percentage uncertainties from trajectory analyses over 10 ns for LiPF₆ and 20 ns for LiFSI using geometry snapshots every 2 ps were found to be 1.8% (0.018 by fraction) for LiPF₆ at 0.26 M and 5.6% (0.056 by fraction) for LiFSI at 2.17 M. These observed uncertainties, due to the solvation structure formation and dissipation dynamics, had negligible impact on the conductance calculations. Analyses lower than 5 ns, typically used in other studies, were not sufficient to cover solvation clusters' full cycle dynamics and cannot lead precise evaluation of the average SSIP fractions.

For LiPF₆, there was nearly complete ion dissociation at 0.14 M, followed by a roughly linear reduction in the fraction of SSIP until 0.9 M. The reduction rate slowed down between 0.9 M to 1.48 M before a sharp decrease to 57.5% occurred at 2.88M. The fraction of SSIP in LiFSI systems started at a much lower value than LiPF₆. This was because LiFSI is a weaker electrolyte than LiPF₆, meaning that ion pairing persisted even at diluted concentrations.

After obtaining a by analyzing the RDF and the SSIP fractions a from the solvation clusters' probability densities, the conductance at infinite dilution Λ_{NE}° was obtained by linear fit applying EQ. 2 using MD simulated conductance Λ_{NE} data. The linear fit for LiPF₆ resulted in an average intercept

$$\frac{1}{{Λ}_{NE}^{°}} = 0.031$$

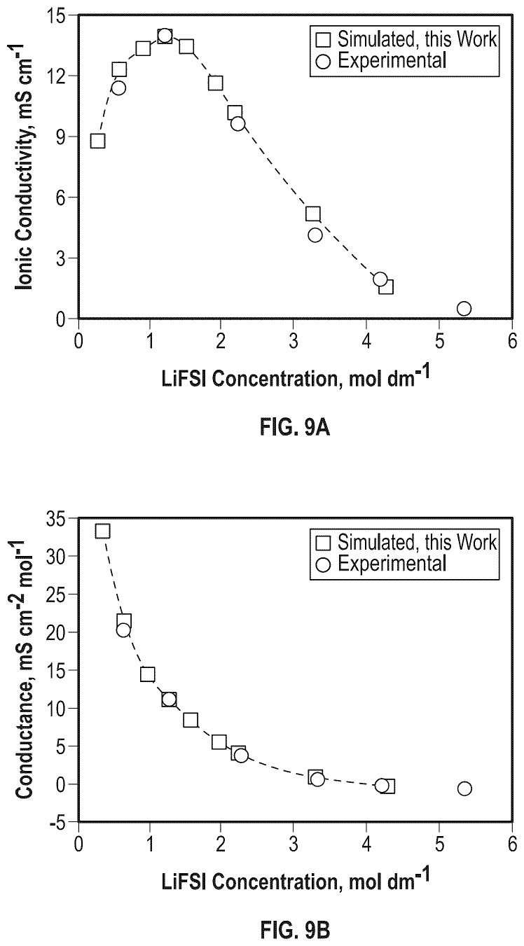

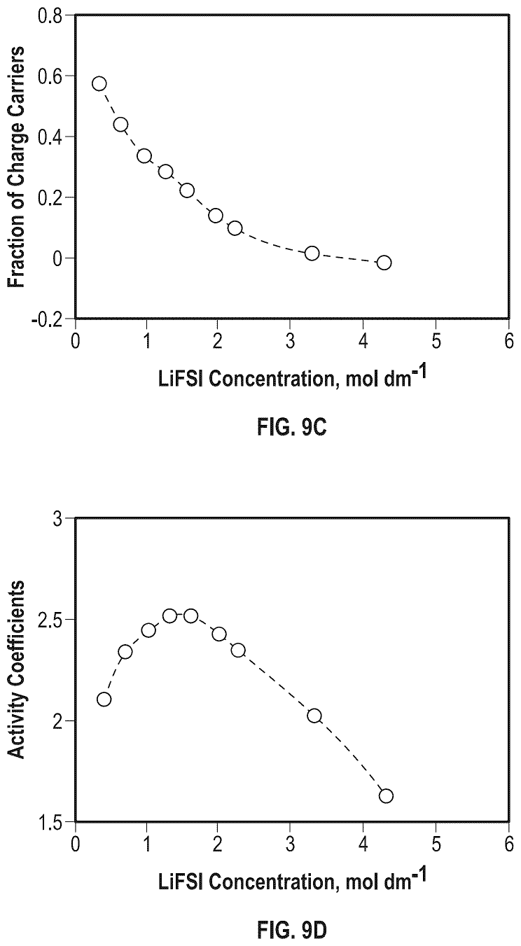

mS⁻¹ cm² mol with a standard deviation of 0.00204 mS⁻¹ cm² mol. The fitting was performed taking into account the uncertainties from statistical analyses and replica simulations using the Scipy ODR package as implemented in the Python programming language. The calculated Λ_{NE}° for LiPF₆ ranged from 30.26 to 34.54 mS·cm⁻² mol⁻¹. Keeping the other parameters fixed, the highest ionic conductivity (σ_{LiPH}^{max} 6) of the best fit (Λ_{NE}°=32.15 mS·cm⁻² mol⁻¹) was 12.2 mS cm⁻¹, about 0.76 mS cm⁻¹ over-estimated than the experimental σ_{LiPH}^{max} 6=11.4 mS cm⁻¹. As shown in FIGS. 8A and 9A, excellent agreement to the experimentally measured ionic conductivity was achieved using the systems and methods of the present disclosure.

FIGS. 8B and 9B show the agreement between the predicted and experimental conductance for LiPF₆ and LiFSI, respectfully. FIGS. 8D and 9D demonstrate the derived activity coefficients for LiPF₆ and LiFSI, respectfully.

For the LiFSI system, a and a parameters were obtained from MD simulations and the conductance at infinite dilution, Λ_{EMP}°, was approximately 105 mS·cm⁻² mol⁻¹. Considering a 5% deviation in Λ_{EMP}°, the calculated bounds enclosed most of the experimental measurements. However, some overestimation was observed below 1M and higher than 2M with respect to the experiments. The highest ionic conductivity was reached at 1.03 M, which generally agreed with both experimental datasets. The predicted ionic conductivity with uncertainty bounds using non-empirically fitted Λ_{NE}° can enclose experimental measurements, however, showed poorer precision compared to those using the empirically fitted Λ_{EMP}° while keeping all other parameters obtained from MD simulations. This comparison between the fully-simulation-predicted and the partially-fitted conductivities indicated the systems and methods of the present disclosure may predict the experimental data with accuracy.

Significant solvation structure speciation differences between these two common chemistries were found. At the salt concentrations which gave the highest conductivities, it was observed that CIPs and small AGGs dominated the LiFSI system, while SSIP dominated the LiPF₆ system. The systems and methods of the present disclosure obtained robust and accurate ionic conductivity predictions for the LiPF₆ system free of empirical fitting. For LiFSI, insufficient precision in conductance sampling at extremely dilute concentrations limited quantitative agreement. This was due to LiFSI's weaker electrolyte nature than LiPF₆ such that complete ion dissociation may not occur at high dilution, leading to challenges in estimating the conductance at infinite dilution. Despite this shortcoming, using the experimental conductance led to quantitative accuracy for LiFSI.

What is claimed is:

1. A method of predicting properties of electrolytes, comprising:
validating at least one molecular force field;
preparing a liquid electrolyte mixture corresponding to said at least one molecular force field;
performing molecular dynamic simulations of said liquid electrolyte mixture;
obtaining simulated molecular dynamic trajectories from said molecular dynamic simulations;
performing molecular structure analysis of said simulated molecular dynamic trajectories; and
predicting ionic conductivity of said liquid electrolyte mixture.

2. The method of claim 1, wherein performing molecular structure analysis comprises:
analyzing liquid structure and ion pairing properties of said molecular dynamic simulations; and
predicting ion pairing thermodynamic quantities of said liquid electrolyte mixture.

3. The method of claim 2, wherein performing molecular structure analysis comprises:
determining at least one type of an at least one molecule;
determining which atoms are bonded to others; and
determining which atoms make up a molecular unit.

4. The method of claim 3, wherein performing molecular structure analysis further comprises:
determining a solvation structure, wherein determining a solvation structure comprises finding all atoms within a radius of a metal cation and anion.

5. The method of claim 4, wherein performing molecular structure analysis further comprises:
identifying at least one aggregate structure; and
resolving the at least one aggregate structure by combining one or more solvation structures if a molecule is shared by the solvation structure of two or more metal cations and anions.

6. The method of claim 2, further comprising:
predicting solubility properties of said liquid electrolyte mixture.

7. The method of claim 1, wherein preparing electrolyte mixture comprises,
mixing at least two solvents to form a mixture;
equilibrating said mixture to form equilibrated solvents; and
adding metal cations and anions to said equilibrated solvents.

8. The method of claim 7, further comprising, performing additional equilibration.

9. The method of claim 1, further comprising:
performing conductance formalism of data obtained from said molecular structure analysis; and
predicting conductance of said liquid electrolyte mixture.

10. The method of claim 1, wherein said molecular dynamic simulations of said liquid electrolyte mixture are performed for at least 10 nano seconds for a strong electrolyte.

11. The method of claim 1, wherein said molecular dynamic simulations of said liquid electrolyte mixture are performed for at least 20 nano seconds for a weak electrolyte.

12. The method of claim 1, wherein said molecular dynamic simulations of said liquid electrolyte mixture are performed for at least 50 nanoseconds.

13. A system for predicting properties of electrolytes, said system comprising:
a database;
a processor; and
a memory storing computer-readable instructions that, when executed by said processor, cause said processor to trigger execution of an electrolyte prediction assistant, wherein said electrolyte prediction assistant comprises:
an automated molecular dynamic simulation module configured to perform molecular dynamic simulations of a liquid electrolyte mixture;
a molecular dynamics analysis module configured to receive molecular dynamic simulations data and analyze ion pairings and associated properties; and
a property prediction module configured to perform conductance formalism of said ion pairings and predict transport, thermodynamics, and solubility properties of said liquid electrolyte mixture.

14. The system of claim 13, wherein said automated molecular dynamic simulation module is further configured to access a database comprising individual molecular force fields.

15. The system of claim 14, wherein said automated molecular dynamic simulation module is further configured to validate said individual molecular force fields.

16. The system of claim 15, wherein said automated molecular dynamic simulation module is further configured to instruct a user to prepare a liquid electrolyte mixture corresponding to at least one validated molecular force field.

17. A computer program product for predicting properties of electrolytes, comprising at least one non-transitory computer readable medium including program instruction that, when executed by at least one processor, cause said at least one processor to:
validate at least one molecular force field;
prepare a liquid electrolyte mixture corresponding to said at least one molecular force field;
perform molecular dynamic simulations of said liquid electrolyte mixture;
obtain simulated molecular dynamic trajectories from said molecular dynamic simulations;
perform molecular structure analysis of said simulated molecular dynamic trajectories; and
predict ionic conductivity of said liquid electrolyte mixture.

18. The computer-program product of claim 17, wherein performing molecular structure analysis comprises:
analyzing liquid structure and ion pairing properties of said molecular dynamic simulations; and
predicting ion pairing thermodynamic quantities of said liquid electrolyte mixture.

19. The computer-program product of claim 17, further comprising:
predict solubility properties of said liquid electrolyte mixture.
